(19) United States
(12) Patent Application Publication
(10) Pub. No.: US 2014/0135925 A1
(43) Pub. Date: May 15, 2014

(54) Breast Reconstruction Device and Methods

(71) Applicant: BioStruxs, LLC, Westlake Village, CA (US)

(72) Inventors: Mai N. Brooks, Westlake Village, CA (US); James P. Watson, Los Angeles, CA (US)

(73) Assignee: BIOSTRUXS, LLC, Westlake Village (US)

(21) Appl. No.: 14/158,855

(22) Filed: Jan. 19, 2014

Related U.S. Application Data
(63) Continuation of application No. 13/098,304, filed on Apr. 29, 2011, now abandoned.
(60) Provisional application No. 61/329,496, filed on Apr. 29, 2010.

(51) Int. Cl. A61F 2/12

(52) U.S. Cl. CPC A61F 2/12
USPC 623/8

(57) ABSTRACT

A novel human breast implant and method for using the same comprising a bioabsorbable implant into which native, autologous vascularized tissue and autologous fat is placed and propagated within a patient's chest as a breast implant.

### RELATED APPLICATIONS

This continuation claims priority pursuant to 35 U.S.C. §120 to patent application Ser. No. 13/098,304, filed Apr. 29, 2011, a U.S. Non-Provisional Patent Application that claims priority to U.S. Provisional Application Ser. No. 61/329,496, filed Apr. 29, 2010, entitled Breast Reconstruction Device and Methods, and is related to the subject matter of the Assignee's U.S. Pat. No. 7,846,728, entitled Tissue Engineering In Vivo With Vascularized Scaffolds, filed Oct. 9, 2007, which claims priority to U.S. Provisional Application Ser. No. 60/851,686, entitled Tissue Engineering In Vivo With Vascularized Scaffolds, filed Oct. 13, 2006, the contents of which are each incorporated in their entirety herein.

### FIELD OF THE INVENTION

The present invention relates to devices and related methods for organ reconstruction and, more particularly, to devices and methods for the reconstruction of breasts.

### BACKGROUND OF THE INVENTION

Breast cancer is the most common form of cancer and the second leading cause of cancer deaths in American women. In 2009, approximately 194,280 patients were estimated to be diagnosed with invasive breast cancer, and an estimated 40,610 will die of this disease (Jemal A., Siegel R., Ward E., Hao Y., Xu J., and Thun M. J., Cancer statistics, 2009. CA Cancer J Clin 2009; 59:225-49; the contents of which are herein incorporated by reference). Furthermore, 62,280 female carcinoma in situ breast cases were diagnosed.

In 2008, according to the American Society of Plastic Surgeons, nearly 79,500 women underwent breast reconstruction surgery post-mastectomy. Approximately 70% of these women had their breast(s) reconstructed with implant(s) whereas the other 30% had autologous breast(s) reconstructed by one of the various flap procedures. Nearly 40% of the implant patients experience severe capsular contracture within ten years. In 2008, more than 14,000 procedures were performed in reconstruction patients to remove the original implants. Complications in breast reconstruction are 2-3 fold higher than in breast augmentation. With a mean follow-up of three years, 36% of breast reconstruction cases needed re-operation compared to 16% of cosmetic cases, versus 22% in revisional cases (Handel N, Cordray T., Gutierrez J., and Hansen J. A., A long term study of outcomes, complications, and patient satisfaction with breast implants, Plast Reconstr Surg 2006; 117:757-67; the contents of which are herein incorporated by reference; the contents of which are herein incorporated by reference). At six years in the Inamed Allergan study, reoperation is required in 52% of reconstruction, 28% in primary augmentation and 40% in revision augmentation (Spear S. L., Murphy D. K., Slicton A., Walker P. S. et al., Inamed silicone breast implant core study results at 6 years, Plast Reconstr Surg 2007; 120:8 S-16S; the contents of which are herein incorporated by reference). The Mentor study showed a two-year rate of complication or reoperation of 43% for primary reconstruction, 42% for revision reconstruction, versus 25% for primary augmentation and 30% for revision augmentation (Cunningham B., The Mentor study on contour profile gel silicone Memory Gel breast implants. Plast Reconstr Surg 2007; 120:33 S-9S; the contents of which are herein incorporated by reference).

Autologous tissue transfer represents a second option for breast reconstruction after mastectomy. The techniques involve free TRAM (transverse rectus abdominis musculocutaneous) flaps, pedicled TRAM, free DIEP (deep inferior epigastric perforator) flaps, pedicled latissimus dorsi myocutaneous flaps, and gluteal flaps. These operations take several hours; require a hospital stay of approximately 4-5 days and subsequent outpatient rehabilitation of approximately 4-6 weeks. The patient then has one or more permanent large scars at the donor site(s). One representative study reports a complication rate of 46%, with 5% total flap loss and 4% partial flap loss (Sullivan S. R., Fletcher D. R. D., Isom C. D., Isik F. F., True incidence of all complications following immediate and delayed breast reconstruction. Plast Reconstr Surg 2008; 122:19-28; the contents of which are herein incorporated by reference). Another recent report showed a complication rate of 27% for TRAM flaps and 68% for latissimus flaps (Spear S. L., Newman M. K., Bedford M. S., Schwartz K. A., Cohen M., and Schwartz J. S., A retrospective analysis of outcomes using three common methods for immediate breast reconstruction. Plast Reconstr Surg 2008; 122:340-7; the contents of which are herein incorporated by reference).

In patients who need radiation, complications are quite common (Jhaveri J. D., Rush S. C., Kostroff K., Derisi D., Farber L. A., Maurer V. E., Bosworth J. L., Clinical Outcomes of Postmastectomy Radiation Therapy after Immediate Breast Reconstruction. Int J Radiat Oncol Biol Phys 2008; 72:859-65; the contents of which are herein incorporated by reference). Complications in this study were scored as follows: Grade 1 (no discomfort), grade 2 (discomfort affecting activities of daily living), grade 3 (surgical interventions or intravenous antibiotics required), and grade 4 (removal or replacement of the reconstruction). The overall rate of severe complications (grade 3-4) was 25%, and rate-of poor functional results was 43%. Acceptable cosmesis was reported by 51% of patients reconstructed with implants and 83% of those reconstructed with autologous tissue reconstruction.

Capsular Contracture

The commercial manufacture of breast implants has existed for over 50 years. Despite many design innovations including foam implants, silicone shells, many different filler materials, foam coating, textured coating, and cohesive gels, capsular contracture still continues to be the most common complication associated with breast implants. No new design innovation on the horizon has shown any promise to prevent this complication from occurring in some patients. The fundamental problem appears to be caused by a nonspecific inflammatory response to a foreign body introduced into human tissue. The Baker classification has continued as the most common standard method to describe contracture (Spear S. L., Baker J. L., Classification of capsular contracture after prosthetic breast reconstruction. Plast Reconstr Surg 1995; 96:1119-23; the contents of which are herein incorporated by reference). This classification rates a breast as follows: Class I—the augmented breast feels as soft as an unoperated one; Class II—the breast is less soft, and the implant can be palpated but it is not visible; Class III—the breast is more firm, the implant can be palpated easily, and it (or distortion from it) can be seen; and Class IV—the breast is firm, hard, tender, painful, cold, and distortion is often marked. Class I and II breasts are considered clinically satisfactory. Class III and IV are considered serious.

The Mentor study with 3-year follow up showed a serious capsular contracture rate of 11% for augmentation and 13% for reconstruction. The Inamed study on silicone implants with data available at six years revealed serious contracture in 16% of reconstruction cases, 15% of cosmetic cases, and 21% in revision augmentation (Spear S. L., Murphy D. K., Slicton A., Walker P. S. et al., Inamed silicone breast implant core study results at 6 years. Plast Reconstr Surg 2007; 120:8 S-16S; the contents of which are herein incorporated by reference). A 10-year study shows contracture rate of 22% for augmentation and 38% for reconstruction, and 42% for revision cases (Handel N., Cordray T., Gutierrez J., and Hansen J. A., A long term study of outcomes, complications, and patient satisfaction with breast implants. Plast Reconstr Surg 2006; 117:757-67; the contents of which are herein incorporated by reference). Contracture is the most common culprit, accounting for 56% of cases that need re-operation. Reporting on a series of 186 implants, Peters et al. observed that Class III-IV contractures continue to accumulate over time, reaching 100% around silicone gel-filled implants at 25 years (Peters W., Smith D., Fornasier V., Lugowski S., Ibanez D., An outcome analysis of 100 women after explantation of silicone gel breast implants. Ann Plast Surg 1997; 39(1):9-19).

Serious capsular contractures require frequent operative interventions such as open capsulotomy, explantation or replacement with new implants. Unfortunately, as seen in the above statistics, revision augmentation cases will have an even higher rate of eventual contractures. Subsequent surgeries will be more difficult, and in many cases will require autologous tissue flaps to preserve the patient's breasts.

Non-Surgical Breast Augmentation

The BRAVA Breast Enhancement and Shaping System is an external tissue expander that is sold on the internet directly to consumers without FDA approval (www.brava.com). The mechanical device is shaped like a bra. Once worn, the system applies a gentle three-dimensional pull, which places the breast under tension. The tension exerted is approximately 15 to 30 mmHg, resulting in fuller breasts over time. The bra should be worn for at least 10 hours a day every day. Side effects include rash, swelling, discomfort, dermatitis, allergic reaction, hyperpigmentation, and costochondritis. A recent study showed that breast enlargement without surgery is possible with this external tissue expander in healthy women with intact breasts without any active breast disease (Schlenz I, Kaider A., The Brava external tissue expander: Is breast enlargement without surgery a reality? Plast Reconstr Surg 2007; 120:1680-9; the contents of which are herein incorporated by reference). Noncompliant subjects were excluded from analysis. The 40 compliant women used Brava 11 hours a day for a median period of 18.5 weeks (range, 14 to 52 weeks). The median volume increase was 155 cc (range, 95 to 300 cc). It is difficult to envision that this device would work on post-mastectomy cases, because there is only skin left without breast in the setting of adjuvant cancer treatments that inhibit new tissue and blood vessel formation.

Fat Grafting Breast Reconstruction

Surgeons have attempted direct autologous fat grafting into the breast for decades (Coleman S. R., Saboeiro A. P., Fat grafting to the breast revisited: safety and efficacy. Plast Reconstr Surg 2007; 119:775-85; the contents of which are herein incorporated by reference). However, tissue resorption often occurs when non-vascularized grafts are transferred in human autograft transplantation. All human autografts undergo this resorption even in the absence of infection, antigen-antibody mismatch, or lack of nutrition. A list of the tissues which have been autografted with well documented resorption over time includes fat grafts. Fat grafts larger than a few mm in diameter are well documented of undergoing resorption over time. Except for small volume fat grafting transferred into multiple well vascularized tunnels, most fat grafts undergo partial resorption.

The most disconcerting aspect is that the resorption rate varies widely from 20 to 90 percent. This makes it difficult to compensate for resorption by overgrafting with larger volumes. When fat is transferred by autologous non-vascularized grafting, by pedicled flaps, or by free microvascular flap transfer, fat necrosis is a minor occurrence with major consequences. Even if only a small percentage of the fat cells undergo cell death, these dead cells undergo saponification releasing abundant long chain fatty acids from the disrupted plasma membrane. Precipitation of these long chain fatty acids with calcium results in palpable masses that appear on mammography to be microcalcifications.

This artifact makes cancer surveillance difficult with mammography. As a consequence, fat grafting for breast augmentation may make future cancer detection difficult. For this reason, autologous fat grafting for cosmetic breast augmentation has been discouraged by the FDA, radiological societies, and the plastic surgery community. Fat necrosis also causes concerns when it occurs in the reconstructed breast. In this scenario, both patients and their oncologists worry that the palpable fat necrosis may be recurrent cancer. This often necessitates a biopsy to rule out this possibility. Fat grafting certainly does not produce enough volume to make an entire breast, therefore it is not a viable option for breast reconstruction.

Fat Stem Cell Breast Reconstruction

Regenerative medicine is a rapidly expanding set of innovative medical technologies that restore function by enabling the body to repair, replace, and regenerate damaged, aging or diseased cells, tissues and organs. There is much interest in the use of both adult and embryonic stem cells. Human adult stem cells have been successfully isolated from liposuction fat (Zuk P A, Zhu M, Ashjian P, et al., Human adipose tissue is a source of multipotent stem cells. Mol Biol Cell 2002; 13:4279-95; the contents of which are herein incorporated by reference).

Several commercial ventures hope to use this fat for breast reconstruction. Cytori Therapeutics developed the Cell-Enhanced Reconstruction, which is a procedure whereby a patient's fat tissue is enriched with his or her own adipose-derived stem and regenerative cells to create a natural filler. Clinical trials are being conducted in Europe in women who underwent lumpectomies for breast cancer. It is difficult to envision that this and similar procedures would effectively provide enough volume to replace one or two entire breasts, as needed for mastectomy cases. Injectable scaffolds are promising substrates for regenerative medicine applications. In a recent study, human adipose-derived stem cells were mixed with multiarm amino-terminated poly(ethylene glycol) (PEG) hydrogels that were crosslinked with genipin, a compound naturally derived from the gardenia fruit (Tan H, Defail A J, Rubin J P, Chu C R, Marra K G., Novel multiarm PEG-based hydrogels for tissue engineering. J Biomed Mater Res A 2009; Epub March 16; the contents of which are herein incorporated by reference). Another material used with stem cells includes porous collagenous microbeads.

Two-Dimensional Meshes and Scaffolds

There are several FDA-approved synthetic bioabsorbable meshes on the market. These are sold as flat sheets of varying sizes, and are indicated for temporary wound or organ support. One example is the commonly used Vicryl mesh made of glycolic and lactic acids (Ethicon). The synthetic materials have been shown to be inert, nonantigenic, nonpyrogenic, and to elicit only a mild tissue reaction during absorption. The knitted mesh has an initial average burst strength of approximately 63 pounds prior to implantation in rats, and retains 80 percent of this strength after 14 days in vivo. Subcutaneous implantation studies in rats indicate that the absorption of Vicryl mesh is minimal until about six weeks, and is essentially complete between 60 and 90 days. Another commonly used mesh is the Dexon brand from U.S. Surgical Corporation. It is constructed from only polyglycolic acid, is inert, nonantigenic, noncollagenous, and does not enhance any secondary infection. For plating, surgeons often use Lactosorb made out of glycolic and lactic acids (Biomet). At initial placement, its strength is comparable to that of titanium plating, and it retains 80 percent of this strength at eight weeks. It is degraded in the human body by hydrolysis completely by one year. Lactosorb is available in a variety of plate shapes and different screw sizes.

AlloDerm (Life Cell) is an acellular dermal matrix derived from donated human skin tissue supplied by US tissue banks. This natural framework consists of proteins with a structurally intact basement membrane, intact collagen fibers and bundles to support tissue ingrowth, intact elastin filaments for biomechanical integrity, and hyaluronan and proteoglycans. Dermagraft (Advanced BioHealing) is manufactured from human fibroblast cells derived from newborn foreskin. These fibroblasts are placed on a bioabsorbable polyglactin mesh. Non-human derived dermal matrix products are also available. For example, Strattice (Life Cell) is derived from porcine dermis and undergoes processing to removes cells and reduce the key component believed to play a major role in the xenogeneic rejection response. Another porcine product is XenMatriX from Brennen Medical.

In general, these two-dimensional meshes and scaffolds are used as adjuncts in breast reconstruction. For example, AlloDerm is sometimes used in implant reconstruction to help enclose the subpectoral pocket and prevent the implant from displacement (Zienowicz R J, Karacaoglu E. Implant-based breast reconstruction with allograft. Plast Reconstr Surg 2007; 120:373-81; the contents of which are herein incorporated by reference). Surgeons may also use bioabsorbable synthetic mesh for the same purpose in the breast and for repair of hernias in flap donor sites.

Three-Dimensional Scaffolds

Scaffold technology has made multilayer tissue engineering possible as well, with multi-cell structures successfully grown in the laboratory. Despite these successes, major roadblocks still exist in translational research. As a consequence, the only human vascular organ successfully engineered to date is a urinary bladder (Atala A., Bauer S. B., Soker S., Yoo J. J., Retik A. B., Tissue-engineered autologous bladders for patients needing cystoplasty. Lancet 2006; 367:1241-6; the contents of which are herein incorporated by reference). Almost all tissue engineering thus far is done with non-vascularized scaffolds. Although neovascularization with capillaries occurs very reliably in scaffolds of about 1 millimeter in thickness, most human organs are much larger than this. As a consequence, tissue engineering on scaffolds is limited in size by the lack of arterial and venous structures which do not grow as well as capillaries. In summary, vascular supply limits organ size in scaffold based tissue engineering.

There has been extensive research by others to develop biocompatible composites/scaffolds. For example, U.S. Publication No. 2002/0022883 to Burg (The contents of which are herein incorporated by reference) described a biocompatible composite with viscous fluid for injection into defects. Of course, this concept would not work for organogenesis. U.S. Publication No. 20040126405 to Sahatjian et al. (The contents of which are herein incorporated by reference) proposed a three dimensional cell scaffold either as a sheet or a tube configured into various shapes. U.S. Pat. No. 5,716,404 to Vacanti et al. (The contents of which are herein incorporated by reference) proposed placing dissociated cells into a biodegradable matrix to be implanted with a tissue expander device into the breast. However, cells would perish without new blood vessels, and this idea did not materialize into practical use since its issued patent in 1998. U.S. Pat. Nos. 5,804,1784, 5,770,193, and 5,759,830 to Vacanti et al. (The contents of which are herein incorporated by reference) also reported the idea of implanting sheets of cell-matrix structure adjacent to mesentery, omentum, or peritoneum tissue.

U.S. Publication No. 2002/0119180 to Yelick et al. (The contents of which are herein incorporated by reference) successfully constructed a biodegradable polymer scaffold molded in the shape of a tooth and placed it onto the omentum of rats. U.S. Publication No. 20030129751 to Grikscheit et al. (The contents of which are herein incorporated by reference) describes a method to achieve high density seeding of polymer scaffold with organoid units. The disclosed scaffolds are collagen-coated 1 centimeter long 0.5 millimeters woven polyglycolic acid tubes with a diameter of 0.5 centimeter, that are sutured to the rat's omentum to make new colonic tissue (Grikscheit T. C., Ochoa E. R., Ramsanahie A., Alsberg E., Mooney D., Whang E. E., Vacanti J. P., Tissue-engineered large intestine resembles native colon with appropriate in vitro physiology and architecture. Ann. Surg. 2003; 238:35-41; the contents of which are herein incorporated by reference).

The Morrison group from Australia developed a vascularized chamber comprising of an empty box which is buried subcutaneously into an animal. An arteriovenous blood leash, either as a ligated pedicle or as an arteriovenous fistulous loop fashioned from the inferior epigastric or femoral vessels into the groin by microsurgical techniques, is inset into the chamber through a small side hole. This sealed ischemic chamber space that cannot close spontaneously promotes an intense and prolonged angiogenic response, and the chamber box fills with granulation tissue. This over time creates a vascularized flap of tissue that can be transplanted to a site of need as a free flap for repair of wound. In the pig, the surgeons included a small amount of autologous living fat into the chamber. Subsequently, 80 milliliters chambers would become filled with tissue of which approximately 50 percent is new fat (Morrison W. A., Progress in tissue engineering of soft tissue and organs. Surgery 2009; 145:127-30; the contents of which are herein incorporated by reference).

In the case of breast reconstruction, immediate reconstruction after mastectomy is a particularly challenging situation for tissue engineering. Almost all women who undergo mastectomy(ies) for breast cancer will need adjuvant systemic therapy shortly afterwards. Chemotherapy reliably suppresses wound vascularization, and anti-hormone drugs such as tamoxifen have been shown to inhibit angiogenesis. Furthermore, a significant number of post-mastectomy patients also need postoperative radiation to the chest and therefore the new “breast.” Radiation effectively eliminates any hope for robust neo-vascularization, not just for the moment but for the rest of the patient's life.

Utilization of the Omentum

The omentum, shown in FIG. 1, has been used by various investigators as a source of vasculature for tissue engineering purposes: porcine tooth (Sumita Y, Honda M J, Ohara T., Tsuchiya S., Sagara H., Kagami H., and Ueda M. Performance of collagen sponge as a 3-D scaffold for tooth-tissue engineering. Biomaterials 2006; 27:3238-48; the contents of which are herein incorporated by reference), dog small intestine (Hori Y, Nakamura T., Matsumoto K., Kurokawa Y., Satomi S., Shimizu Y., Tissue engineering of the small intestine by acellular collagen sponge scaffold grafting. Int. J. Artif. Organs. 2001; 24:50-4; the contents of which are herein incorporated by reference), dog tracheal defects (Kim J., Suh S. W., Shin J. Y., Kim J. H., Choi Y. S., Kim H., Replacement of a tracheal defect with a tissue-engineered prosthesis: early results from animal experiments. J. Thorac. Cardiovasc. Surg. 2004; 128:124-9; the contents of which are herein incorporated by reference), canine oral epithelial cells and rib chondrocytes (Suh et al., 2004), and porcine bladder urothelial cells (Moriya K., Kakizaki H., Murakumo M., Watanabe S., Chen Q., Nonomura K., Koyanagi T., Creation of luminal tissue covered with urothelium by implantation of cultured urothelial cells into the peritoneal cavity. J Urol. 2003; 170:2480-5; the contents of which are herein incorporated by reference).

Recently, a successful human clinical trial has been reported (Atala A., Bauer S. B., Soker S., Yoo J. J., Retik A. B., Tissue-engineered autologous bladders for patients needing cystoplasty. Lancet 2006; 367:1241-6; and U.S. Publication No. 2007/0059293 to Atala; the contents of which are herein incorporated by reference). Autologous bladder cells were seeded on a biodegradable bladder-shaped scaffold made of collagen and polyglycolic acid, which was then implanted covered with omentum into the patients with myelomeningocele. In all of the above studies, the omentum was used as a single layer attached to one side of a flat scaffold, or wrapped around a three-dimensional scaffold.

The Vacanti group also used the mesentery and interscapular fat pad to grow hepatocytes, intestinal cells and pancreatic islet cells in mice and rats (Vacanti J. P., Morse M. A., Saltzman W. M., Domb A. J., Perez-Atayde A., and Langer R., Selective cell transplantation using bioabsorbable artificial polymers as matrices. J. Pediatr. Surg. 1988; 23:3-9; the contents of which are herein incorporated by reference).

In clinical practice, the omentum flaps have been used most commonly for chest wall reconstruction after sternal dehiscence. Omental flaps have rarely been harvested laparoscopically for direct breast reconstruction (Zaha H., Inamine S., Naito T., and Nomura H., Laparoscopically harvested omental flap for immediate breast reconstruction. Am J Surg 2006; 192:556-8; the contents of which are herein incorporated by reference). To allow passage of the omentum, a subcutaneous tunnel was made from the medial end of the inframammary fold to the xiphoid process. The omentum was stapled to the pectoralis major muscle and then manually shaped into a mound. The mastectomy skin was then closed. Unlike TRAM or DIEP flaps, the omental flap method leaves minimal scars from the harvesting procedure. The operative time is shorter than with traditional TRAM and DIEP flaps. Hospital stay would be much shorter, potentially just overnight, because the patient does not need to recover from extensive musculocutaneous dissection and/or close monitoring required for free flaps.

However, some major disadvantages have prevented omental flaps from wide clinical acceptance. The shape of the resulting reconstructed breast can widely vary due to lack of structural support. The size of the omentum is variable in individuals, and there is no reliable method prior to surgery to accurately estimate its size. Usually, the omentum is too small to adequately reconstruct both breasts and sometimes is too small to reconstruct even only one breast. Interestingly, an implant placed in this situation surrounded by omentum has a much less likelihood of developing contracture (Cothier-Savay I., Tamtawi B., Dohnt F., Raulo Y., Baruch J., Immediate breast reconstruction using a laparoscopically harvested omental flap. Plast Reconstr Surg 2001; 107:1156-63; the contents of which are herein incorporated by reference).

What is needed in the art is a breast implant that overcomes the above described shortcomings of the prior art.

### OBJECTS AND SUMMARY OF THE INVENTION

The present invention is a novel human breast implant and method for using the same that addresses many of the shortcomings of the prior art implants by providing a bioabsorbable implant into which autologous vascularized tissue and autologous fat is placed within the patient in order to propagate vascularized tissue that is subsequently transferred to the patient's breast as a breast implant.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a perspective view of an omentum.

FIG. 2 is a side elevation view of an implant according to one embodiment of the present invention.

FIG. 3A is a plan view of an implant according to one embodiment of the present invention.

FIG. 3B is a perspective view of an implant according to one embodiment of the present invention.

FIG. 4 is a perspective view of a base of an implant according to one embodiment of the present invention.

FIG. 5 is a perspective view of a body of an implant according to one embodiment of the present invention.

FIG. 6 is a partial cut-away view of an implant according to one embodiment of the present invention.

FIG. 7 is a plan view of a body of an implant according to one embodiment of the present invention.

FIG. 8 is a side elevation view of a body of an implant according to one embodiment of the present invention.

FIG. 9A is a side elevation view of a body of an implant according to one embodiment of the present invention.

FIG. 9B is a side elevation view of a body of an implant according to one embodiment of the present invention.

FIG. 10 is a perspective view of a body of an implant according to one embodiment of the present invention.

FIG. 11 is a side elevation view of a base of an implant according to one embodiment of the present invention.

FIG. 12 is a plan view of a base of an implant according to one embodiment of the present invention.

FIG. 13 is a perspective view of a base of an implant according to one embodiment of the present invention.

FIG. 14 is a cut-away side elevation view according to one embodiment of the present invention.

FIG. 15 is a partial transparent perspective view of an implant according to one embodiment of the present invention.

FIG. 16 is a perspective view of a base of an implant according to one embodiment of the present invention.

FIG. 17 is a partial transparent perspective view of an implant according to one embodiment of the present invention.

FIG. 18 is a perspective view of a base of an implant according to one embodiment of the present invention.

FIG. 19 is a side elevation view of a base of an implant according to one embodiment of the present invention.

FIG. 20 is a side elevation view of a base of an implant according to one embodiment of the present invention.

FIG. 21 is a chemical formula of a co-polymer employed in an implant according to one embodiment of the present invention.

FIG. 22 is a perspective view of the finite element analysis mesh of a body of an implant according to one embodiment of the present invention.

FIG. 23 is a perspective view of the finite element analysis of a body of an implant according to one embodiment of the present invention.

FIG. 24 is a flow diagram of a method for using an implant according to one embodiment of the present invention.

FIG. 25 is a photograph of a 20× magnification of a portion of a stained slide of vascularized rat fat produced according to one embodiment of the present invention.

FIG. 26 is a photograph of a 100× magnification of a portion of a stained slide of vascularized rat fat produced according to one embodiment of the present invention.

FIG. 27 is a photograph of an x-ray of an implant removed from a pig that was produced according to one embodiment of the present invention.

FIG. 28 is a photograph of a portion of fat produced in a pig according to one embodiment of the present invention.

FIG. 29 is a photograph of a magnification of a portion of a stained slide of vascularized pig fat produced according to one embodiment of the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present invention represents a novel solution for organ reconstruction and, specifically, for breast reconstruction that is based, in part, on a bioabsorbable implant scaffold. Broadly speaking, the device of the present invention employs a bioabsorbable scaffolding which houses an autologous, natively vascularized tissue and an autologous, fat tissue matrix. For example, the bioabsorbable scaffold may include an outer shell formed in the shape of a breast. The autologous, natively vascularized tissue may, for example, include at least a portion of the omentum.

The device and associated methods of use of the present invention provide numerous advantages over current devices and methods for breast reconstruction. For example, the bioabsorbable scaffold of the present invention is tissue-based without a permanent foreign body thereby avoiding the associated high complications and re-operation rate described above for typical implants. The operative time for employing the bioabsorbable scaffold of the present invention is a few hours, shorter than traditional flap reconstruction, and blood loss is significantly less, thus stress to the patient's body is minimized. The postoperative monitor requirements after employing the bioabsorbable scaffold of the present invention are less intensive than that for free flaps. Furthermore, the hospital stay length is similar to implant reconstruction, 1-2 days, versus 4-6 days required for traditional flap reconstruction. Finally, because there is no permanent muscle loss, no large scars and potential defects at the flap donor site(s), rehabilitation subsequent to employing the bioabsorbable scaffold of the present invention is anticipated to be many weeks less than traditional flap reconstruction.

In order to overcome the limitations of organ size in scaffold-based tissue engineering due to limited vascular supply, the present invention employs a vascularized scaffold having a complete vascular bed 5. Preferably, an autologous, natively vascularized tissue provides this vascular bed 5. The vascular bed 5 ideally comprises at least one large inflow artery of, for example, approximately 2-3 millimeters and at least one large outflow vein of, for example, approximately 3-4 millimeters connected to a native circulatory network.

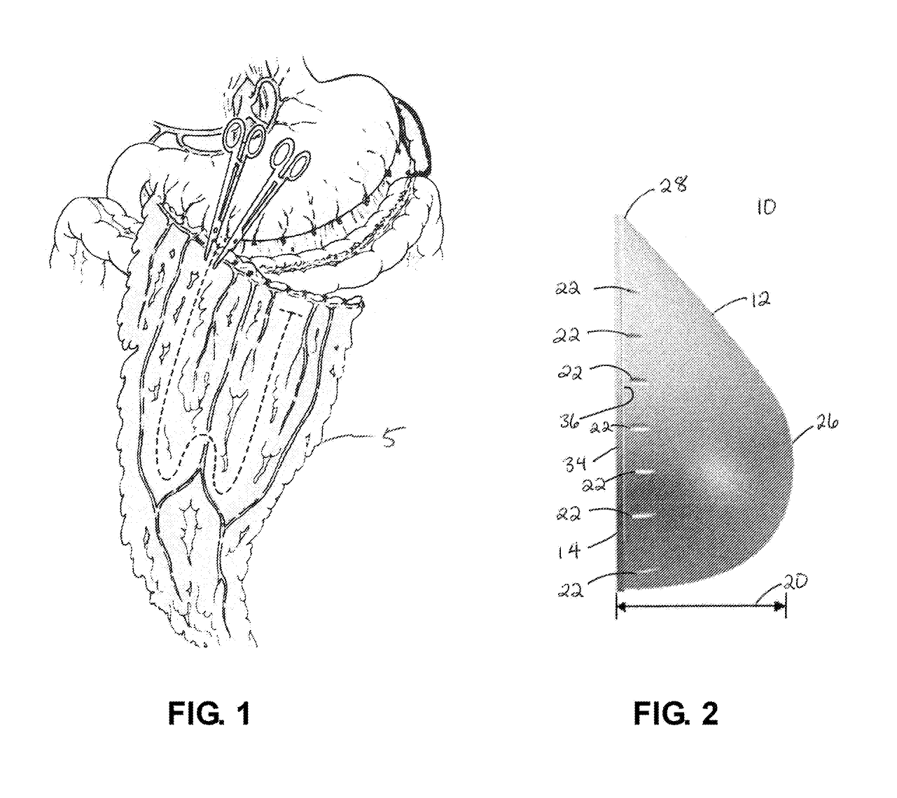

In one embodiment of the present invention, the omentum, shown in FIG. 1, is employed for the vascular bed. The omentum contains its own rich vascular supply, both arterial and venous, throughout its structure. The large arteries branch into hundreds of arterioles and capillaries, the large veins into hundreds of venules. These vessels interdigitate and interconnect in a complex three-dimension network.

An omentum-based vascularized growth chamber has significant promise for many translational research applications. Rationale for this optimism includes the following reasons. First, the omentum is a naturally occurring, expendable vascular scaffold that has been shown to develop a rich capillary network. Despite the fact that it is only fat, lymphatics and blood vessels, it has been used to revascularize ischemic areas, treat lymphedema, and cover the heart after sternal debridements. Second, the omentum has a dual blood supply. This means that it can be split into two separate sections thereby allowing for develop two vascularized growth chambers in the extra-peritoneal space. This would make it ideal for breast reconstruction tissue engineering, since two breasts could be made. In this model, the omentum retrieval could be done using laparoscopic techniques. Then, the growth chamber would be inserted under the breast skin and above the pectoralis muscle.

In certain other embodiments, the device of the present invention employs sources of vascular bed 5 other than the omentum. For example, in one embodiment, a partial rectus abdominis muscle may be employed for the vascular bed 5. In this embodiment, a small incision is made to harvest a small amount of the muscle, instead of the entire muscle in the traditional TRAM (transverse rectus abdominis musculocutaneous) flap procedure. Other contemplated sources for the vascular bed 5 include, but are not limited to, a partial, or entire, latissimus muscle; a partial, or entire, external oblique muscle; and a partial, or entire, serratus anterior muscle.

With respect to the non-biological structure or scaffold of the present invention, the outer shape of the bioabsorbable scaffold is designed to resemble that of a human breast. The scaffold interior is configured to accommodate multiple layers or folds of the vascular bed 5, for example multiple folds of an omentum that is placed within the scaffold in contact with an autologous fat tissue matrix.

With reference to FIGS. 2-6, in one embodiment of the present invention, a bioabsorbable implant 10 includes a bowl-shaped body 12 and a base 14. The implant 10 may, for example have a volume of approximately 270 cubic centimeters with a width 16 of approximately 12 centimeters, a height 18 of approximately 10.1 centimeters, and a depth 20 of approximately 4.8 centimeters.

The body 12 includes a tapered distal end 26 and a broad proximal rim 28. The proximal rim 28 defining an opening into an interior 32 of the body 12. The body 12 has a substantially hollow interior 32 having a plurality of baffles 24. The body 12 includes, for example, 7 baffles 24 that are approximately parallel to one another and span the width 16 of the implant 10. The baffles may, for example have a thickness of 2 millimeters. The baffles 22 initiate on the interior surface of the interior 32 at the distal side of the body 12 and extend towards the proximal rim 28. In order to allow room for the folds of the vascular bed and fat cell matrix, all or a portion of the baffles 24 do not extend completely to the proximal rim 28. Stated alternatively, the baffles 24 are recessed within the interior 32 of the body 12 relative to the rim 28.

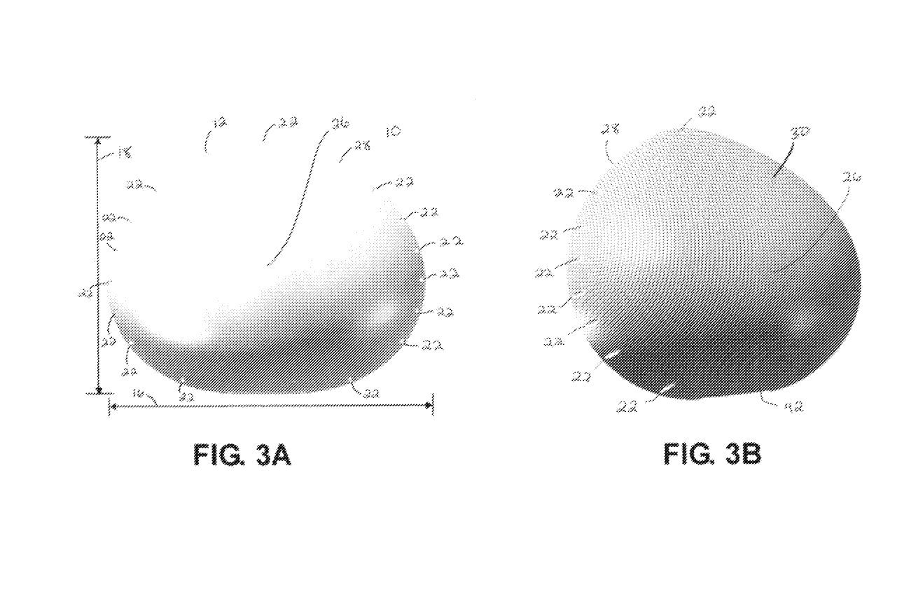

As shown in FIG. 3B, in certain embodiments of the present invention, the body 12 includes perforations 30 that serve to facilitate fluid transfer through the implant 10. The perforations 30 may be evenly dispersed over or across body 12 and/or may be dispersed relative to the position of the baffles 24 or other structural feature(s). In FIG. 3B, the perforations 30 are shown as lines dispersed across the body 12.

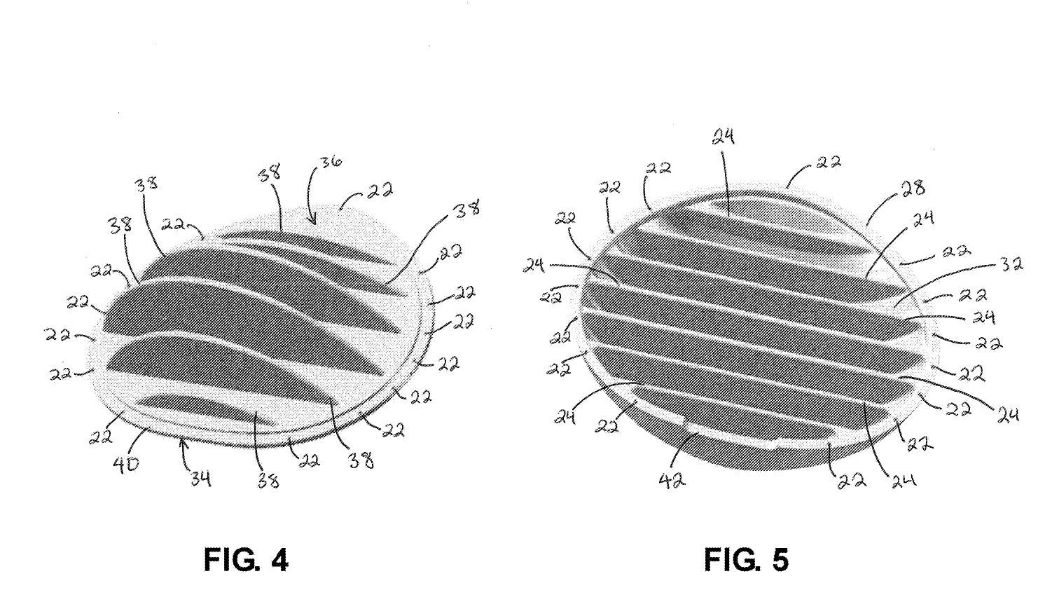

With reference to FIG. 4 base 14 is substantially solid and includes a proximal side 34, a distal side 36, and a plurality of fins 38. The base 14 may further employ a recess 40 around all or a portion of the periphery of the distal side 36 of the base 14. The recess 40 provides a surface that is complementary to the proximal rim 28 of the body 12. The base includes, for example 6 fins 38 that initiate in the distal side 36 of the base 14 and extend outward distally from the distal side 36 of the base 14. The fins 38 have a rounded profile when viewed in cross-section, such as shown in FIG. 4. The fins 38 of the base 14 are approximately parallel to one another and span the width 16 of the body 12. The fins 38 are spaced relative to one another and to the height 18 of the implant 10 so as to insert between the baffles 24 of the body 12 when the body 12 and the base 14 are mated or brought together to form the implant 10.

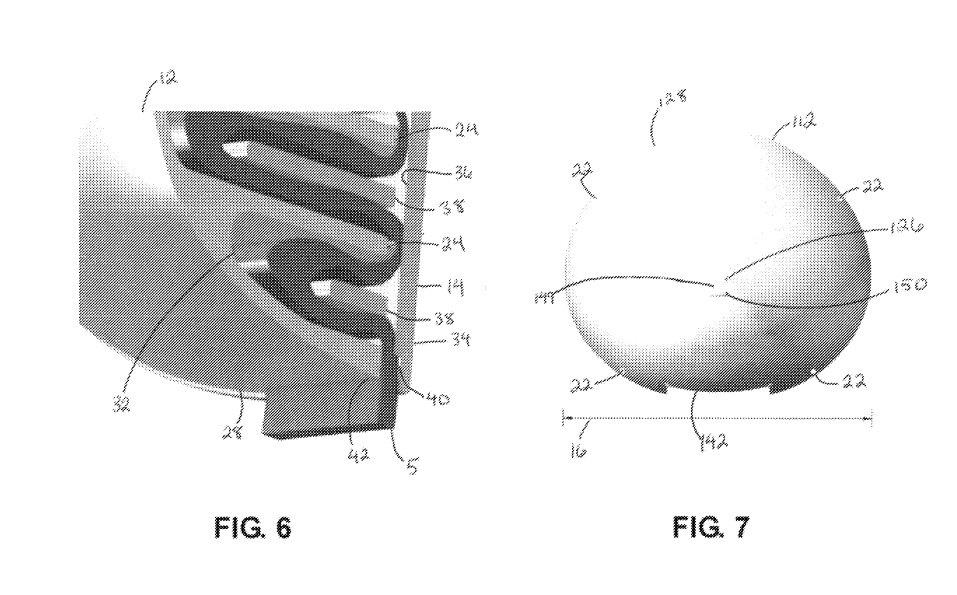

FIG. 6 shows a partial cut-away view of the body 12 mated to the base 14 with the vascular bed 5 interposed between the baffles 24 of the body 12 and the fins 38 of the base 14. As shown in FIGS. 3B, 5 and 6, the body 12 further comprises aperture 42. Aperture 42 is formed by a notch or recess in the proximal rim 28 and the recess 40 of the base 14. The aperture serves as a port through which the vascular bed 5, and thus the circulatory network of the vascular bed 5, may be enter and exit the implant 10. While the aperture 42 is shown as being positioned at a bottom portion of the implant, it is contemplated that the aperture 42 may be positioned at alternative locations and that more than one aperture 42 may be employed in an implant 10.

Formed within the body 12 and base 14, proximate an outer periphery of the body 12 and base 16 are suture points 22. The suture points 22 of the body 12 are positioned complementary to the suture points 22 formed within the base 14. The suture points 22 may comprise holes through the body 12 and base 14 or may comprise regions of the body 12 and base 14 that are thinner than the surrounding structure. The suture points 22 allow for the easy attachment of the body 12 to the base 14 using known suturing methods.

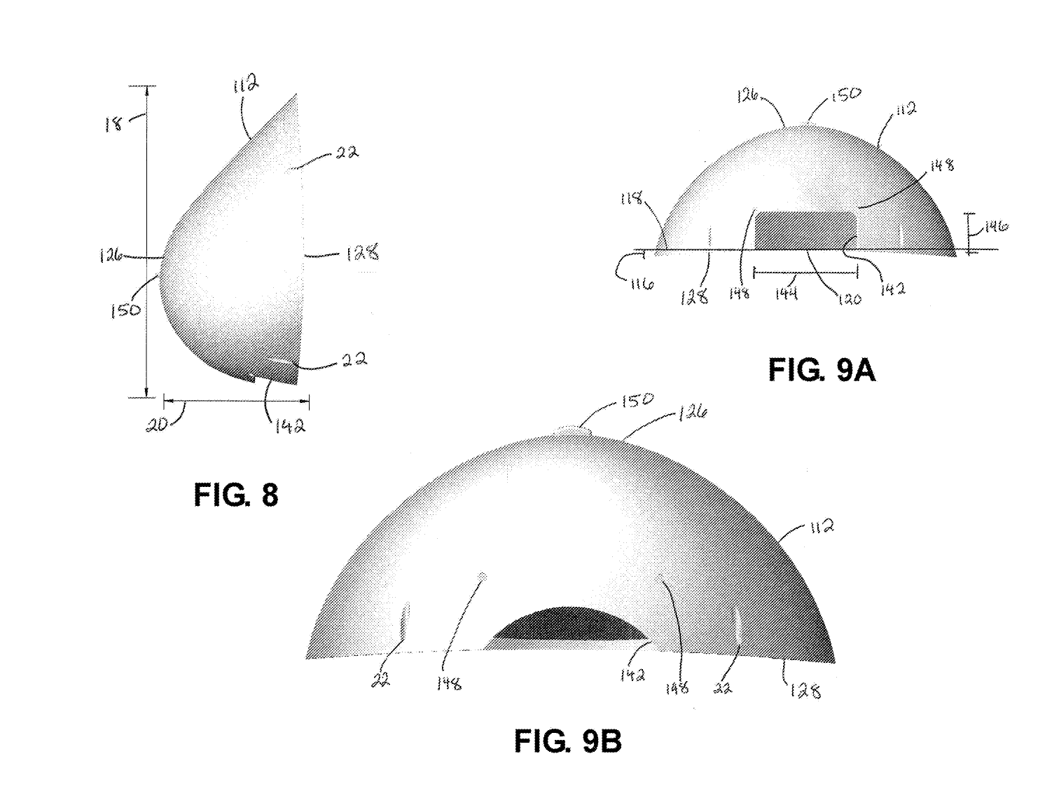

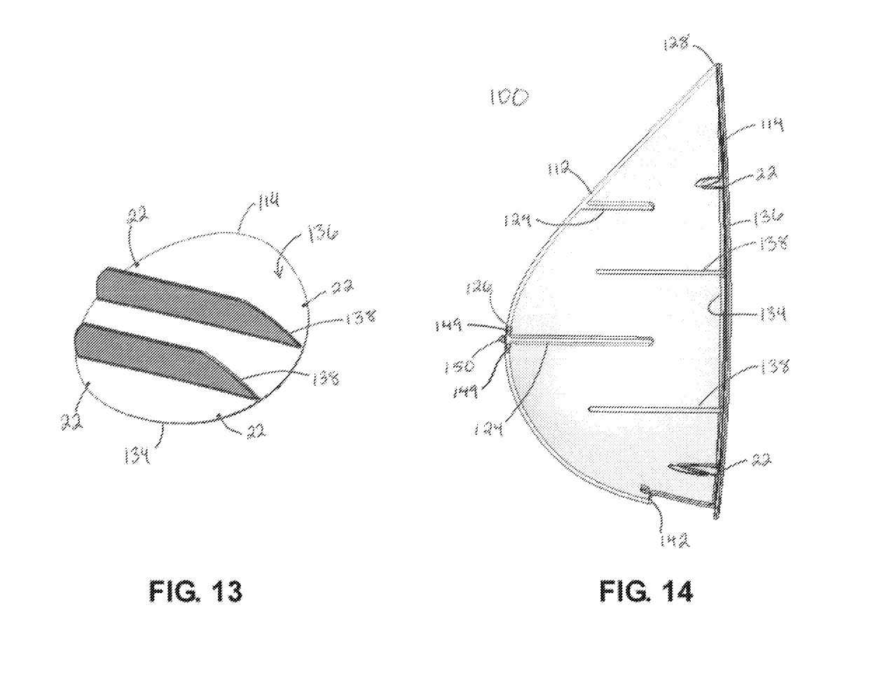

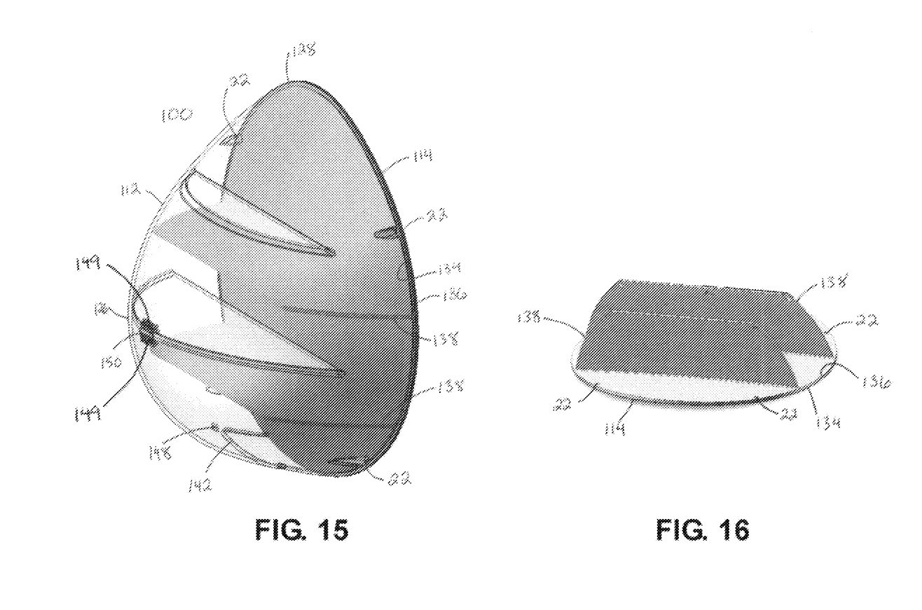

With reference to FIGS. 7-16, in another embodiment of the present invention, the implant 100 is similar to the above described implant 10 with the following exceptions. The implant 100 has, for example, a volume of approximately 300 cubic centimeters with a width 16 of approximately 12 centimeters, a height 18 of approximately 11 centimeters, and a depth 20 of approximately 5.2 centimeters.

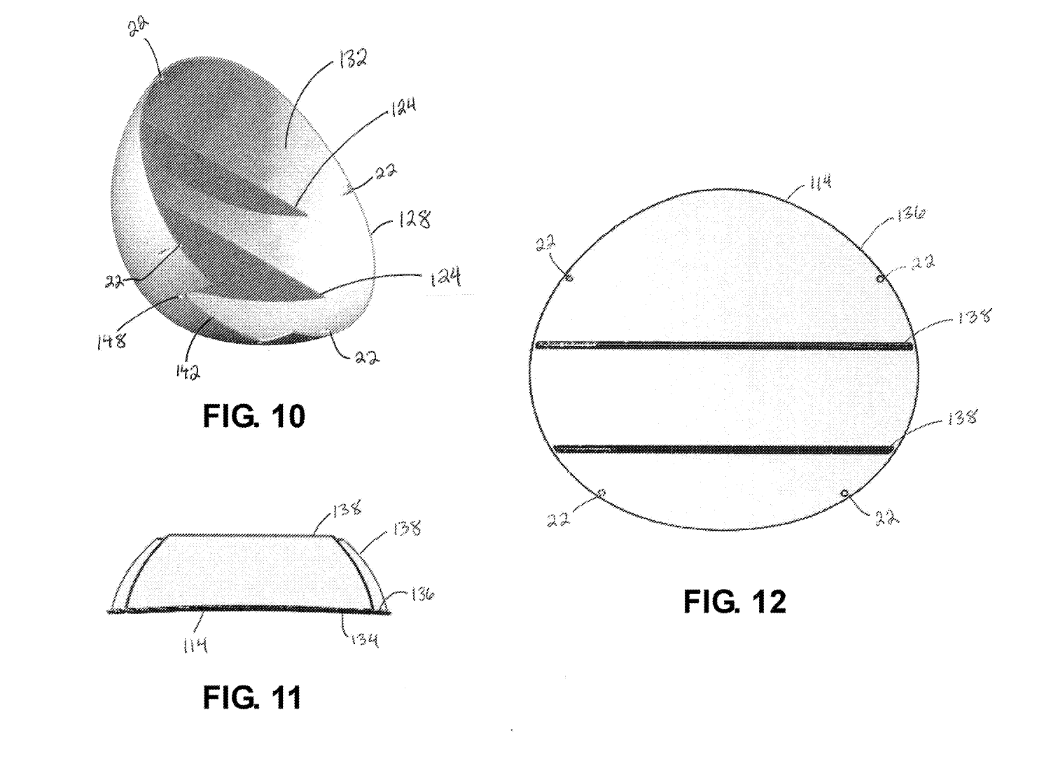

The proximal rim 128 of the body 112 of the implant 100 is irregularly formed such that the proximal rim 128 follows the contour of a human chest. For example, the rim 128 is formed such that it is not planar when viewed in cross-section, such as in FIG. 10. FIG. 10 shows that the left side of the rim 128 from an angle 116 of approximately 5 degrees beyond a planar line 118 drawn through a midpoint of the body 120 shown in FIGS. 9A and 9B. In this manner the rim 128 wraps around the contour of the chest of the patient more so than a planar rim 28. Furthermore, as shown in FIG. 10A, the body 112 employs a rectangular aperture 142 that has a width 144 of approximately 4 centimeters and a depth 14 of approximately 1.5 centimeters. As shown in FIG. 9B, in an alternative embodiment, the body 112 employs a rounded or arched aperture 142. Proximate the aperture 142, the body 112 employs one or more holes 148 through which the vascular bed 5 may be secured to the body 112 by, for example, suturing the vascular bed 5 to the body 112.

With reference to FIGS. 7, 9, 14, and 15, body 112 further employs ports 149 through which injections into or extractions out from the implant 100 are made. For example, the ports 149 may comprise a plurality of holes or perforations located proximate the distal end 126 of the body 112. The ports 149 may be used to, for example, inject additional fat tissue matrix, nutrients, or pharmaceuticals into the implant 100 after the implant 100 has been implanted. In order to assist the physician or other caregiver in locating the ports 149, the ports 149 are positioned proximate a marker 150. The marker 150 may, for example, have an elevated form such as a nipple.

As shown in FIGS. 10, 14, and 15, the body 112 of the implant 100 employs only two baffles 124. Also of note is that the baffles 124 are recessed further within the interior 132 of the body 112 than the baffles 24 of the body 12. These features of the implant 100 may advantageously provide increased space for the vascular bed 5 and fat tissue matrix within the implant 100.

As shown in FIGS. 11-16, the base 114 employs 2 fins 138. The fins 138 initiate in the distal side 138 of the base 114 and extend outward such as the fins 38 described above for implant 10, however, the fins 138 have a truncated, flat profile when viewed in cross-section, such as shown in FIGS. 11, 13, and 15.

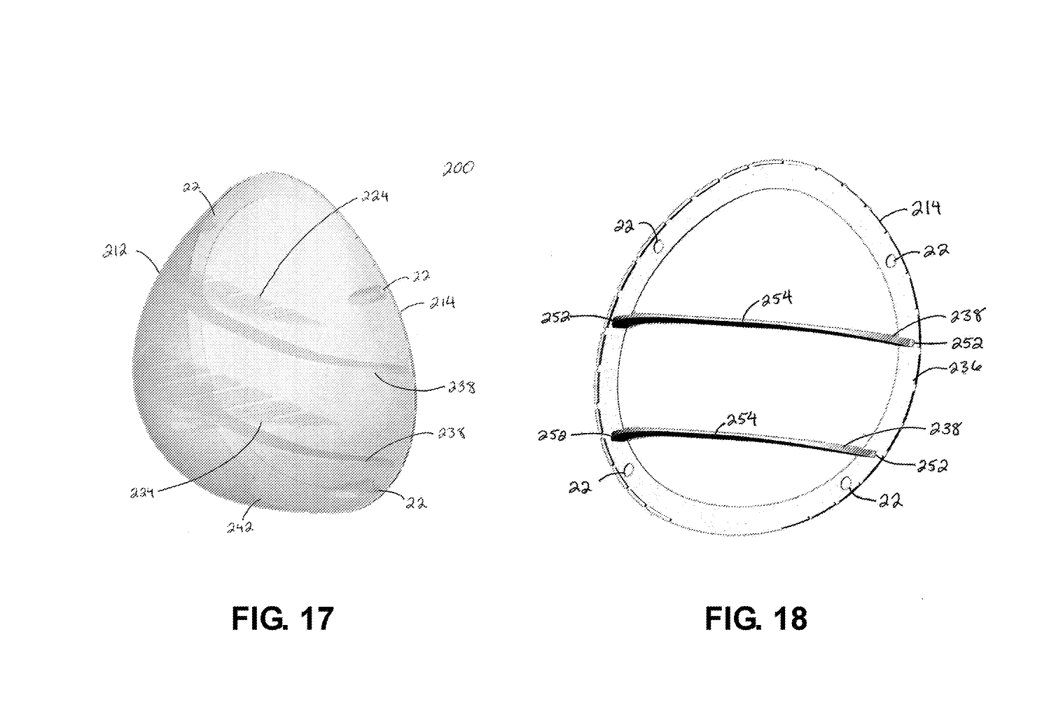

With reference to FIGS. 17-20, in another embodiment of the present invention, the implant 200 is similar to the above described implant 10 and 100 with the following exceptions. The implant 200 employs a body 212 that includes baffles 224 that are slotted or otherwise non-solid in form.

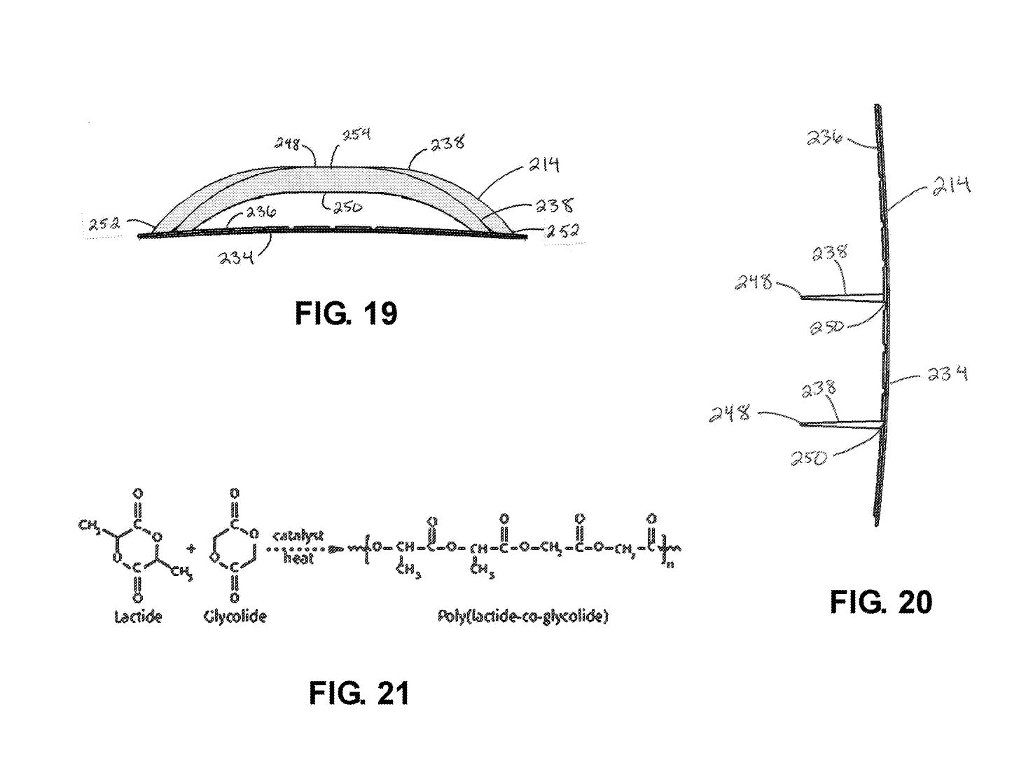

In addition, implant 200 employs a base 214 that has an annular form instead of the solid forms described above with respect to the bases 14 and 114. The base 214 includes two fins 238 that initiate from the distal side 238 of the base 114, however, given the annular form of the base 214, i.e. the absence of material in the central portion of the distal side 236 of the base 214, the fins 238 initiate from the distal side 236 only at a left and right side 152 of the base 214. As shown in FIG. 19, the fins 238 employ an arched or rounded distal side 248 and proximal side 250. As shown in FIG. 20, the fins 238 further employ a tapered profile in which the proximal sides 250 of the fins 228 are thicker than the distal sides 248. As apparent from FIG. 18 the fins 238 are also tapered across their width. Stated alternatively, the fins 238 are thicker at the right and left sides 252 than the mid-portions 254. Finally, in contrast to the recess 40 of base 14, as shown in FIG. 18, base 214 employs an elevation. When the base 214 and the body 212 are mated, the elevation extends around an outside periphery of the body 212 and serves to align and stabilize the mated base 214 to the body 212.

The implants 10, 100, and 200 according to the present invention may be formed of a single or a combination of bioasorbable material. The various components of the implants 10, 100, and 200, e.g. the body, baffles, base, and fins, may be formed of the same or different bioasorbable material. In certain embodiments, the various components of the implants 10, 100, and 200 are formed by injection molding techniques known in the art. Following injection molding, the components of the implants are subjected to gamma irradiation for sterilization.

Exemplary bioabsorbable polymers that may be employed to form implants according to the present invention include, but are not limited to, polylactic acid (PLA), polyglycolic acid (PGA), and mixtures thereof. By utilizing polyglycolide and poly(l-lactide) as starting materials, as shown in FIG. 21, it is possible to co-polymerize the two monomers to extend the range of homopolymer properties.

These polymers are medical grade materials with excellent safety profiles, used in a wide range of medical devices, and are produced, for example by Boehringer Ingelheim Resomer of Ridgefield, Conn. These polymers degrade through non-enzymatic hydrolysis of the ester bonds. This degradation occurs as follows: (1) water penetrates the implant, attacking the chemical bonds and breaking the polymer chains (hydrolysis); (2) hydrolysis converts the long chains into non-toxic natural metabolites (lactic acid and glycolic acid); (3) these molecules are metabolized by the liver into CO₂ and H₂O and released through the lungs (Middleton J., Tipton A., Synthetic Biodegradable Polymers as Medical Implants. In: Medical Plastics and Biomaterials, 1998; the contents of which are herein incorporated by reference).

Table 1 below summarizes the material properties of three co-polymers that may be employed to form the implants of the present invention.

TABLE 1
Material Physical Properties

| | | Stress | Modulus of |
|---|---|---|---|
| | Inherent | at load, | Elasticity, |
| Bioresorbable Polymer | Viscosity | MPa | MPa |
| Poly(L-lactide-co-D,L-lactide) 70:30 | 3.8 | 73 | 432 |
| Poly(L-lactide-co-glycolide) 85:15 | 6.0 | 87 | 424 |
| Poly(L-lactide-co-glycolide) 82:18 | 2.1 | 87* | 424* |

The inherent viscosity also referred to as intrinsic viscosity or i.v., is provided in deciliters per grams as 0.1 percent in CHCl₃, at 25 degrees Celsius. It is also noted that the physical properties of the 82:18 polymer are comparable to the published values of the 85:15 polymer.

In order to access the physical properties of the bodies 12, 112, 212, static finite element analysis of the poly(L-lactide-co-glycolide) 85:15 body 12 was performed. Static finite element analysis, FEA, is a numerical simulation technique used to calculate and visualize stresses and displacements of a material structure under load. A linear finite element simulation was performed on the body 12. A 145 pound load was applied to a small region of the distal end of body 12 of the model. The following material properties were used for the analysis:

| | | Modulus of |
|---|---|---|
| | Tensile Modulus, | Elasticity, |
| Bioresorbable Polymer | MPa | MPa |
| Poly(L-lactide-co-glycolide) 85:15 | 87 (12618 psi) | 424 (61496 psi) |

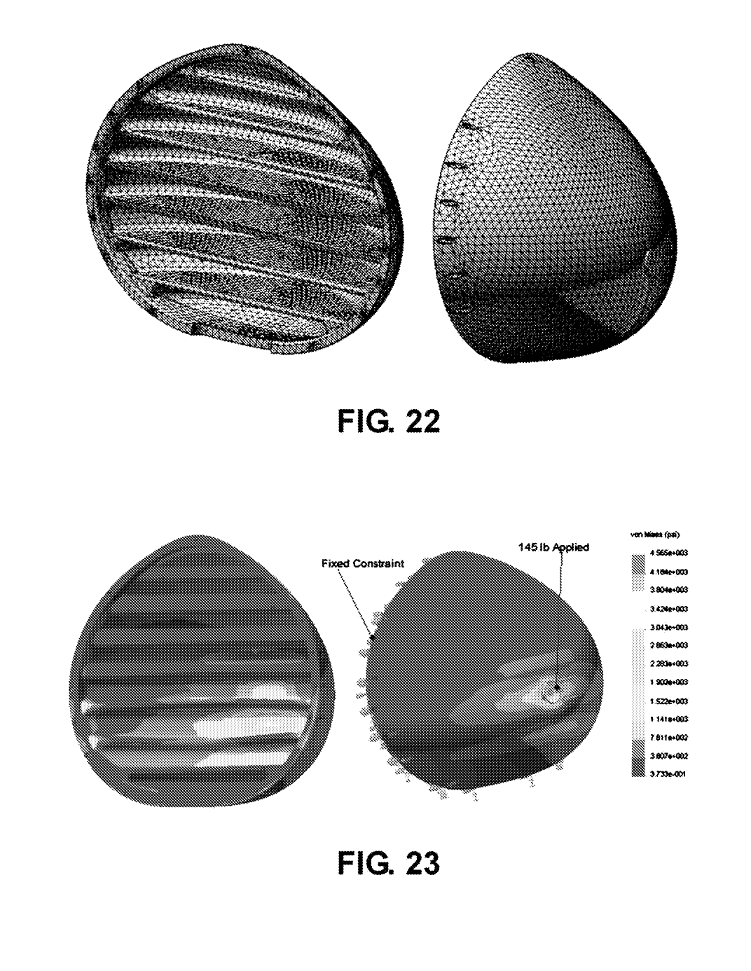

The maximum stress developed for the 145 pound load condition was 4,565 pounds per square inch, psi. This value is well below the tensile strength 12,618 psi of the material. Accordingly, the body should be operable to preserve the shape of the breast against gravity and the pressure of overlying post-mastectomy skin. 145 pounds represents the expected stress delivered upon the body 12 when the patient weighing up to 300 pounds lies prone, putting her mid-body weight on the implant 10. In general, most morbidly obese women are not candidates for breast reconstruction. FIG. 22 shows the FEA mesh applied to the model body 12, and FIG. 23 shows the results of the FEA.

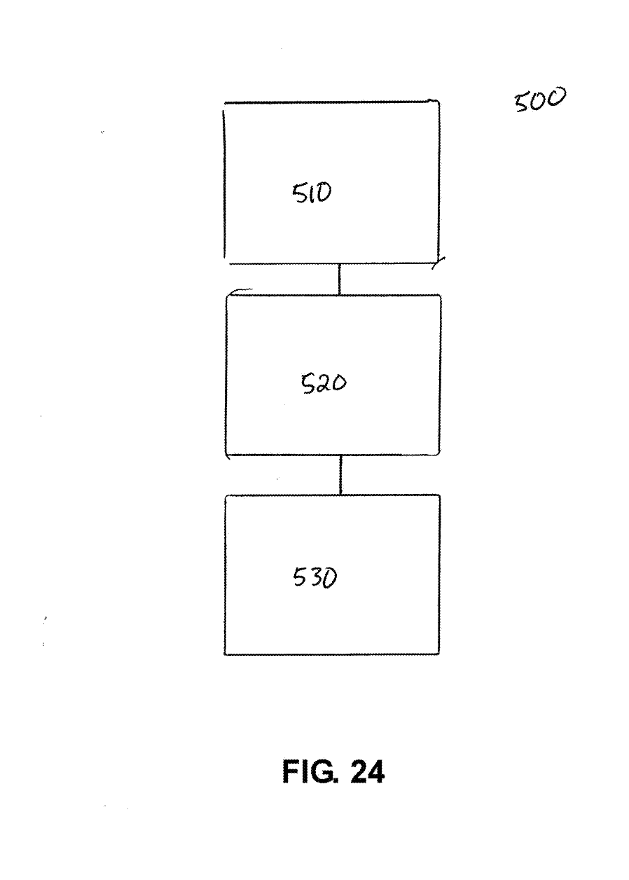

Turning next to a method of use for the above described implants, FIG. 24 illustrates a generalized flow-diagram of the steps employed in a method 500 for using the bioabsorbable implants according to the present invention. The method 500 comprises a first step 510 in which fat is harvested from the patient by liposuction. Step 520 includes the identification and isolation of the vascular bed 5, for example the native omentum, within the chest defect of the patient by, for example, formation of a subcutaneous tunnel. Other sources of the vascular bed 5 include the rectus abdominis muscle, latissimus muscle, external oblique muscle, or serratus anterior muscle.

Next, the method 500 comprises step 530 in which the implant is assembled within the patient at the location of the chest defect that resulted from the mastectomy. The step 30 includes packing the isolated omentum inside the implant along with the fat tissue matrix, and suturing the implant body to the implant base by utilizing known suturing methods and the suture points 22 formed within the body and base of the implant. In order to maintain blood flow into and out from the assembled implant, a portion of the vascular bed 5 containing arterial and venous vasculature is placed though the aperture 42, 142, or 242 and maintained in vascular communication with the patient's native circulatory network. Alternatively, the body and base of the implant are sewn together with running sutures, leaving just enough space at the intersection of the body and base to allow blood flow into and out from the vascular bed 5, but to prevent the fat tissue matrix from drifting or migrating out of the implant.

The following experimental examples were performed to further access the present invention.

### EXAMPLE 1

### Rat Study

Preliminary experiments were carried out in Sprague Dawley female rats at approximately 3.5 months in age. Under general anesthesia, an incision was made in the inguinal region of the rat, and a portion of the rat's fat tissue was harvested. This fat tissue was manually mixed with PuraMatrix (Becton Dickinson) in 10 percent sucrose solution. The fat tissue matrix or mixture was placed inside a biodegradable mesh pocket fabricated of Dexon mesh (U.S. Surgical Corporation), and secured shut with sutures. A midline laparotomy incision was made in the same individual rat, its omentum was identified and wrapped around the mesh pocket, and secured with sutures. The rats tolerated the surgery well, and recovered without any complications. Four weeks later, the rats were sacrificed. The mesh pockets with fat inside were placed in paraffin, and Hematoxylin & Eosin, H&E, stained slides were generated.

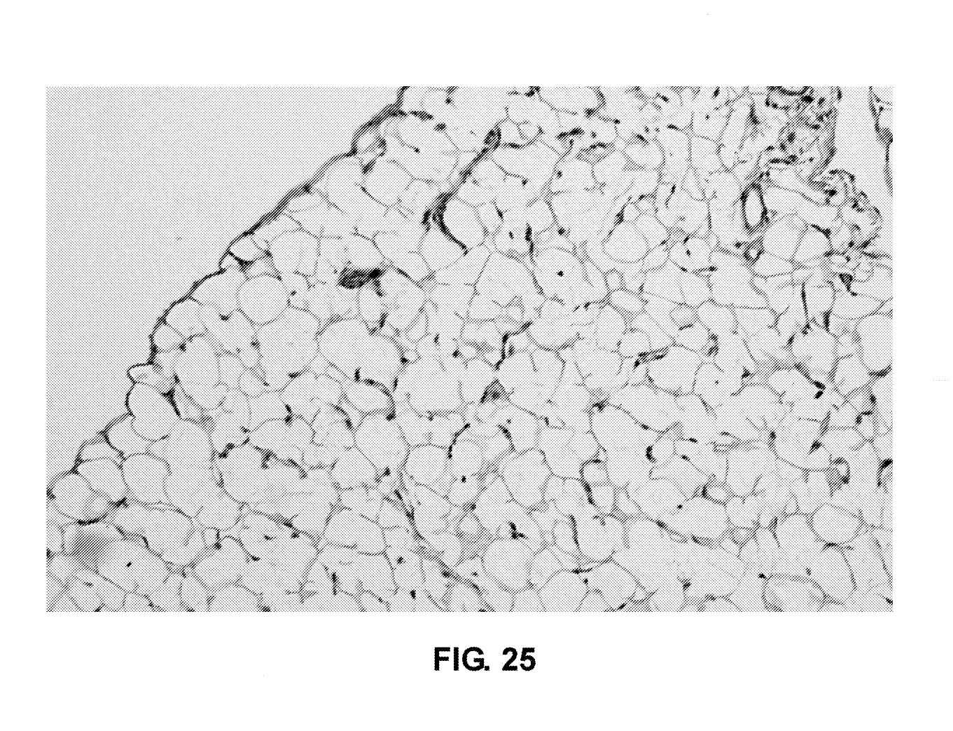

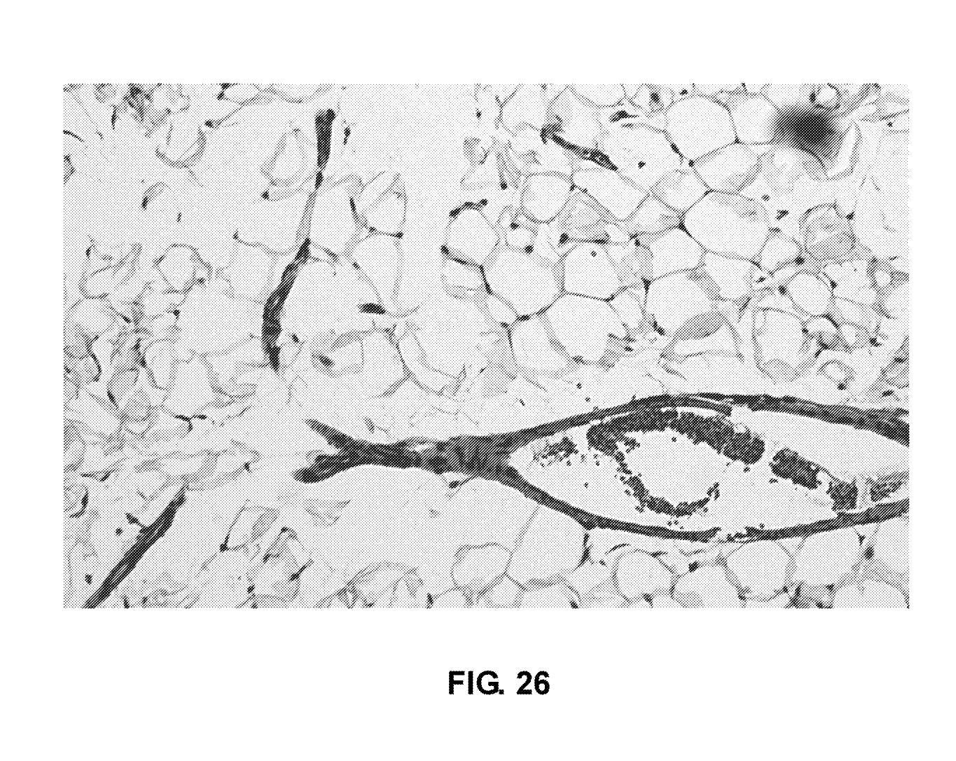

The results demonstrated that the fat tissue inside the mesh pocket survived and was well vascularized. The thickness of the fat tissue ranges from 2-6 millimeters. FIGS. 25 and 26 show well vascularized fat tissue at 20× and 100× magnification, respectively.

In other rat-based experiments, the harvested fat tissue was placed immediately adjacent to the omentum, and the mesh was wrapped outside both fat and omentum. H&E histochemistry demonstrated that both fat and omentum were incorporated into well vascularized fatty tissue with thickness ranging from 4-10 millimeters at four weeks.

### EXAMPLE 2

### Pig Study

For a pig study, a 9-month old Yucatan female pig weighing 50 kg was used. Under general anesthesia, an incision was made in the subcutaneous abdominal region of the pig, and a portion of the pig's fat tissue was harvested. A midline laparotomy incision was made, the omentum identified, and placed inside the scaffold. The fat tissue matrix was placed inside the implant between the folds of the omental, and the base and body of the implant was secured shut with sutures. The implant remained inside the pig's abdominal cavity, attached to the omentum blood supply. The laparotomy incision was closed with running sutures.

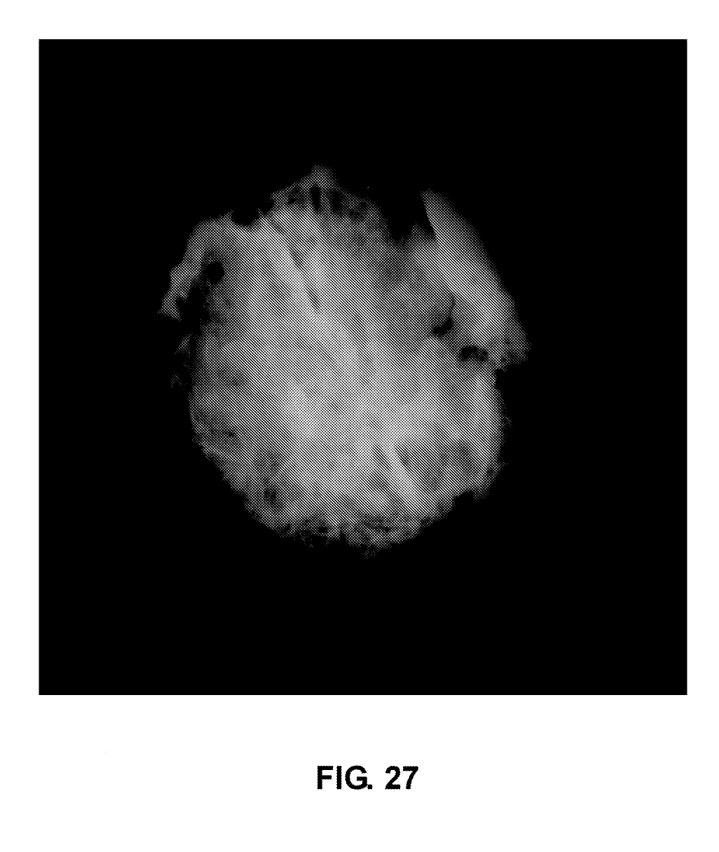

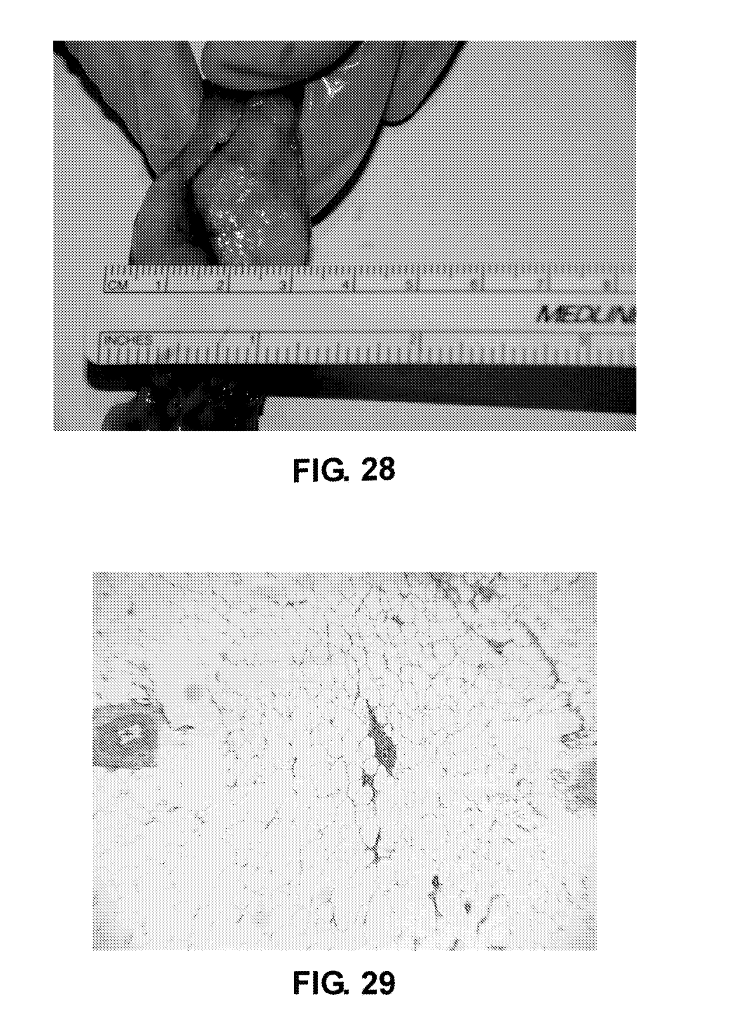

The pig was observed during recovery daily by veterinary staff and exhibited no signs of complications. Four weeks later, the pig was sacrificed. The time period of four weeks was chosen because it usually takes at least two weeks to develop histological evidence of fat necrosis. The implant with omentum and fat inside was retrieved. In the breast, fat necrosis may manifest as calcifications seen on mammograms. Therefore, the scaffold was x-rayed, and as shown in FIG. 27, no calcium was observed. As shown in FIG. 28, grossly, it appeared that the new fat grew to a layer as thick as 1.5 centimeters. No gross evidence of tissue necrosis was observed when the implant was sectioned.

Subsequently, the tissue inside the implant was sectioned, preserved in paraffin, and H&E stained slides of approximately 5 micrometer were generated. Under the microscope, as shown in FIG. 29, no fat necrosis inside the implant was observed. We also performed histological examinations of the pig omentum (outside the scaffold), liver, spleen, intestine, and skin. All of these tissues and organs exhibited no sign of damage or toxicity under the microscope.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An implant comprising: a bioabsorbable body having a bowl-like profile including a tapered end, a rim defining an opening into an interior of the body, and a plurality of baffles spanning across the interior of the body, and a base having a boundary substantially complementary to the rim of the body and a plurality of fins extending outward from the base; wherein when the rim of the body is mated with the base the fins of the base extend into the interior of the body and between the baffles of the body.

2. The implant of claim 1, wherein the implant is a breast implant.

3. The implant of claim 1, wherein the body further comprises a plurality of suture points.

4. The implant of claim 1, wherein the body comprises perforations.

5. The implant of claim 1, wherein the rim of the body comprises a recess that forms an aperture into the interior of the body when the body is mated with the base.

6. The implant of claim 1, wherein a distal edge of at least one fin is rounded.

7. The implant of claim 1, wherein a proximal edge of at least one fin is rounded.

8. The implant of claim 1, wherein along a width of at least one fin the thickness of the fin varies.

9. The implant of claim 1, wherein the implant comprises a body having two baffles and a base having two fins.

10. The implant of claim 1, wherein at least one baffle is slotted.

11. The implant of claim 1, wherein the implant comprises a bioabsorbable polymer.

12. The implant of claim 1, wherein the implant comprises a co-polymer formed from at least glycolide and lactide.

13. An implant comprising: a bioabsorbable body having breast-shaped form and an aperture;
and a vascular bed a portion of which is positioned within an interior of the body and a different portion of which extends through said aperture.

14. The implant of claim 13, wherein the bioabsorbable body comprises a co-polymer formed from at least glycolide and lactide.

15. The implant of claim 13, wherein the bioabsorbable body comprises a plurality of apertures.

16. The implant of claim 13, wherein the interior of the body comprises a fat tissue matrix.

17. The implant of claim 13, wherein the interior of the body comprises a plurality of structures about which the vascular bed is folded.

18. The implant of the claim 13, wherein the portion of the vascular bed that extends through the apertures comprises an arterial structure.

19. A breast implant comprising: comprising a bioabsorbable body and further comprising an annular base having a plurality of elements spanning from one side to the base to another side of the base; and a body having the form of a human breast and a rim that mates with the base.

20. The breast implant of claim 19, wherein when the base is mated to the body the plurality of elements of the base are interposed between baffles within an interior of the body.
